(19) United States
(12) Patent Application Publication
(10) Pub. No.: US 2023/0330379 A1
(43) Pub. Date: Oct. 19, 2023

(54) DEVICE AND METHOD FOR MITIGATING AEROSOL RELEASE FROM NEBULIZATION OR RESPIRATORY SYSTEM

(71) Applicants: ARIZONA BOARD OF REGENTS ON BEHALF OF ARIZONA STATE UNIVERSITY, SCOTTSDALE, AZ (US); MAYO FOUNDATION FOR MEDICAL EDUCATION AND RESEARCH, ROCHESTER, MN (US)

(72) Inventors: Erica Forzani, Phoenix, AZ (US); Xiaojun Xian, Sioux Falls, ND (US); Bhavesh Patel, Scottsdale, AZ (US); Kelly McKay, Southern Pines, NC (US)

(21) Appl. No.: 18/043,525

(22) PCT Filed: Sep. 1, 2021
(86) PCT No.: PCT/US21/48619

§ 371 Date: Feb. 28, 2023

Related U.S. Application Data
(60) Provisional application No. 63/073,437, filed on Sep. 1, 2020.

(51) Int. Cl. A61M 16/10; A61M 16/06

(52) U.S. Cl. CPC A61M 16/1065; A61M 16/0683; A61M 16/0666; A61M 16/0616; A61M 16/1055; A61M 2205/7509; A61M 2205/7518

(57) ABSTRACT

An apparatus and method for mitigating release of aerosols from a nebulizer treatment device, or from the respiratory system of a contaminated subject utilizes a mask configured to cover the nose and mouth of the subject, and utilizes a high efficiency filter and fan coupled with an exhaust port of the mask. The fan creates a pressure differential that causes the respiratory products to flow through the high efficiency filter, where aerosols are captured. The mask may further include at least one inlet port for admission of gas (e.g., oxygen) or aerosols. The apparatus provides adequate inhaled oxygen levels and avoids carbon dioxide rebreathing problems.

### CROSS-REFERENCE TO RELATED APPLICATIONS

This application claims priority to U.S. Provisional Patent Application No. 63/073,437 filed on Sep. 1, 2020, wherein the entire contents of the foregoing application are hereby incorporated by reference herein.

### BACKGROUND

Airborne diseases are transmitted by the spread of microorganisms (also referred to as microbes) mainly through aerosols and micro droplets. Contaminated micro droplets are frequently generated by an infected host through sneezing, coughing, breathing, speaking and sweating. Airborne diseases not only affect human health, but also detrimentally impact the global economy.

Aerosols are microscopic particles of 0.01 um to 100 um in size suspended in air. Ninety-nine percent of aerosols produced by humans (regardless of age, sex, weight, and height) are less than 10 um. The small size of most aerosols produced by humans is concerning, since smaller aerosols take longer to settle than larger ones and are therefore more likely to be inhaled into the lungs of other individuals. In a turbulent atmosphere, aerosols of 100 μm take an average of 5.8 seconds to settle on surfaces, while 0.5 μm aerosols may take 41 hours to settle. If aerosols contain viable pathogens, they can be a threat while airborne and even after they settle on surfaces since they can generate elements that are sources of contamination. In the case of SARS-CoV-2, viruses can be viable on a surface for up to two days.

Nebulization treatments provide an effective means of treatment for respiratory diseases. A nebulizer is used to turn liquid medicine into a very fine mist (which may embody a vapor) that can be inhaled by a patient through a face mask or mouthpiece. Unfortunately, nebulization treatment creates a high risk of spreading pathogens due to the nature of the therapy. Aerosol portions that do not reach the alveolar area of a patient's lungs remain in the dead volume of the patient's respiratory system (including the nose and mouth) in contact with infectious areas, and are subsequently exhaled into the environment as contaminated aerosols.

The dispersion of aerosols and results of potential mitigation techniques are difficult to quantify, since aerosol spreading patterns can be dependent on air exchange rates and ventilation streams of a room, the nebulization mechanism, environmental conditions (e.g. temperature and humidity), charge of the aerosol, and exposure period and location of individuals. Because of the almost unpredictable difficulty of predicting aerosol patterns, it is essential to mitigate the dispersion of aerosols from contaminated sources. The art continues to seek improvement in devices and methods for mitigating airborne contamination due to aerosol release from respiratory systems (e.g., respiring users) and nebulizers used for respiratory treatment.

### SUMMARY

The present disclosure relates to an apparatus and method for reducing aerosol release from a nebulizer treatment device or the respiratory system of a contaminated source due to coughing, sneezing, or using vocal cords. A mask is configured to cover the nose and mouth of a user, and includes at the least an oxygen supply port with or without an aerosol supply port, and an exhaust port. Oxygen supply is provided in the aerosol supply port or in a separate port. Aerosols (e.g., medication) is supplied from a nebulizer to the aerosol supply port, and aerosol products exhaled by the user are received from the exhaust port. A high efficiency filter, and a fan are arranged downstream of the exhaust port. The fan creates a pressure differential that causes the respiratory products to flow through the high efficiency filter, where exhaled aerosols are captured, thereby reducing or eliminating the release of contaminated aerosols from the user. The apparatus assures adequate inhaled oxygen levels and avoids carbon dioxide rebreathing problems.

In one aspect, the disclosure relates to an aerosol mitigation apparatus comprising oxygen supply port without an aerosol supply port. The apparatus comprises: a mask comprising a compliant portion configured to conform to portions of a face of a user and cover the nose and mouth of the user; an oxygen supply port associated with the mask and configured supply oxygen from a source to the mask to be inhaled by the user; an exhaust port associated with the mask and configured to receive respiratory products exhaled by the user; a high efficiency filter arranged downstream of the exhaust port to capture the respiratory products exhaled by the user; and a fan configured to promote flow of aerosol products exhaled by the user.

In another aspect, the disclosure relates to an aerosol mitigation apparatus. The apparatus comprises: a mask comprising a compliant portion configured to conform to portions of a face of a user and cover a nose and mouth of the user; an oxygen supply port and an aerosol supply port associated with the mask and configured supply aerosols from a nebulizer to the mask to be inhaled by the user; an exhaust port associated with the mask and configured to receive respiratory products exhaled by the user; a hygroscopic condenser humidifier and a filter arranged downstream of the exhaust port to capture respiratory products exhaled by the user; and a fan configured to promote flow of respiratory products exhaled by the user from the exhaust port through the hygroscopic condenser humidifier and the filter.

In certain embodiments, the high efficiency filter is integrated with the hygroscopic condenser humidifier as a hygroscopic condenser humidifier filter.

In certain embodiments, the compliant portion comprises silicone (e.g., to improve sealing).

In certain embodiments, the fan is arranged either (A) downstream of the high efficiency filter, or (B) between (i) the exhaust port and (ii) the hygroscopic condenser humidifier and the filter.

In certain embodiments, the apparatus further comprises a flexible hose extending between the exhaust port and the fan.

In certain embodiments, the apparatus further comprises a portable battery pack or portable power supply coupled with the fan.

In certain embodiments, the apparatus further comprises an aerosol supply port associated with the mask and configured to supply aerosols from a nebulizer to the mask to be inhaled by the user.

In certain embodiments, the apparatus further comprises a breath actuated nebulizer associated with the mask and configured to supply aerosols to the mask to be inhaled by the user.

In certain embodiments, the apparatus further comprises a nasal cannula integrated with the mask and configured to receive aerosols from the aerosol supply port.

In certain embodiments, the apparatus further comprises a head strap coupled with the mask and configured to secure the mask to a face of the user.

In certain embodiments, the apparatus further comprises a T-joint coupled with the mask, wherein the aerosol supply port and the exhaust port are arranged at two legs of the T-joint.

In another aspect, the disclosure relates to a method for reducing aerosol release from a respiring user. The method comprises: supplying oxygen from at least one supply port associated with a mask, wherein the mask comprises a compliant portion configured to conform to portions of a face of the user and cover a nose and mouth of the user, and the oxygen is to be inhaled by the user through the mask; and receiving aerosol products exhaled by the user from an exhaust port associated with the mask, and creating a pressure differential using a fan to cause the aerosol products to flow to a high efficiency filter arranged downstream of the exhaust port.

In another aspect, the disclosure relates to a method for reducing aerosol release from a nebulizer treatment device. The method comprises: supplying aerosols from a nebulizer to an aerosol supply port associated with a mask, wherein the mask comprises a compliant portion configured to conform to portions of a face of a user and cover a nose and mouth of the user, and aerosols are to be inhaled by the user through an aerosol supply port associated with the mask; and receiving respiratory products exhaled by the user from an exhaust port associated with the mask, and creating a pressure differential using a fan to cause the respiratory products to flow through a hygroscopic condenser humidifier and a filter arranged downstream of the exhaust port. In certain embodiments, the nebulizer comprises a breath actuated nebulizer associated with the mask.

In another aspect, any of various aspects and/or features described herein may be combined for additional advantage.

### BRIEF DESCRIPTION OF DRAWINGS

FIG. 1 is a schematic diagram showing how droplets and aerosols may be propagated from an infected host (e.g., a SARS-CoV-2 infected host) to a susceptible host in an environment.

FIG. 2 is a plot of particle concentration versus time showing particle counts sensed at three different distances (3, 6, and 9 feet, respectively) from a patient undergoing nebulizer therapy.

FIG. 3A is a front view of an aerosol mitigation apparatus useable for nebulization treatment according to one embodiment of the present disclosure.

FIG. 3B depicts a front portion of a nebulization treatment apparatus including a mask according to the design shown in FIG. 3A, modified to provide a nasal cannula and omitting a flexible hose.

FIG. 3C depicts a rear portion of the nebulization treatment apparatus of FIG. 3B.

FIG. 4A depicts a nebulization treatment apparatus including a full-face mask with a hygroscopic condenser humidifier having an integrated bacterial/viral filter as part of a bilevel positive airway pressure (BPAP) device.

FIG. 4B depicts a human subject wearing the apparatus of FIG. 4A.

FIG. 5A is a side view of a human test subject wearing a High Flow Nasal Cannula (HFNC) useful as a nebulization treatment apparatus and covered by a surgical mask.

FIG. 5B is a front view of the human test subject and HFNC of FIG. 5A, without presence of the surgical mask.

FIG. 5C is a front view of the human test subject wearing the HFNC and surgical mask shown in FIG. 5A.

FIG. 6A is perspective view of a portion of a nebulization treatment apparatus according to one embodiment including a silicone mask modified to incorporate a nasal cannula according to the design shown in FIGS. 3B and 3C.

FIG. 6B is a perspective view of a human test subject wearing the nebulization treatment apparatus of FIG. 6A, showing a portable battery pack powered fan arranged between (i) an exhaust port of the mask and (ii) a HCH and integrated high efficiency filter.

FIGS. 7A and 7B provide perspective views of a subject connected to a regular nebulization treatment apparatus according to one embodiment corresponding generally to the design of FIG. 3A, but without a flexible hose arranged between the fan and mask exhaust port.

FIGS. 8A and 8B show a mannequin fitted with a Non-Invasive Positive Pressure Ventilator (NIPPV), arranged in a mitigation box having a bacterial/viral filter coupled to a port defined in the box.

FIG. 9A is a magnified front perspective view of a full face mask portion of the NIPPV fitted over a head of the mannequin as shown in FIGS. 8A-8B.

FIG. 9B is a side view of the face mask, mitigation box, and filter of FIGS. 8A-8B.

FIG. 10 is a diagram showing placement of sensing modules having particle counters at different distances of 3, 6, and 13 feet, respectively, from a subject undergoing nebulizer treatment.

FIG. 11 is a bar chart summarizing efficacy of different mitigation methods that were applied to the different oxygen therapies and the type of nebulizers according to Cases I to V.

FIGS. 12A-12C provide plots of aerosol particles per cubic foot versus time sensed at different distances from the subject for Case I.

FIGS. 13A-13C provide plots of aerosol particles per cubic foot versus time sensed at different distances from the subject for Case II.

FIGS. 14A-14C provide plots of aerosol particles per cubic foot versus time sensed at different distances from the subject for Case III.

FIGS. 15A-15C provide plots of aerosol particles per cubic foot versus time sensed at a different distances from the subject for Case IV.

FIGS. 16A-16C provide plots of aerosol particles per cubic foot versus time sensed at different distances from the subject for Case V.

FIG. 17 shows an aerosol mitigation apparatus including a mask, designed for transportation of a patient, the mask having an associated fan and high efficiency filter coupled to an outlet port, and including an oxygen port for admission of medical oxygen.

FIG. 18 provides plots of aerosol particles per cubic feet versus time sensed at different distances from a subject wearing a system like the one shown in FIG. 17, for three different conditions.

FIG. 19 is a bar chart showing the inhaled carbon dioxide levels of the aerosol mitigation mask system of FIG. 17 for four different conditions.

FIG. 20 is a perspective view of nebulization treatment apparatus providing aerosol barrier utility, with the apparatus including a fan and high efficiency filter coupled to an outlet port of a face mask, and including a breath-actuated nebulizer coupled with an inlet port for admission of aerosols. The

FIG. 21A is a bar chart comparing amounts of saline physiological solution intake (mL intake per minute) between an aerosol barrier nebulizer and an unmodified nebulizer.

FIG. 21B is a bar chart comparing absolute solution intake comparing absolute solution intake amount (in osmolarity units) per minute assessed in an inhalation filter.

### DETAILED DESCRIPTION

An apparatus and method for reducing aerosol release from a nebulizer treatment device or the respiratory system of a contaminated source (e.g., a respiring user, which may be subject to aerosol ejecting activities such as coughing, sneezing, or vocalization) are provided. A mask includes an oxygen supply port, an aerosol supply port, and an exhaust port, and is configured to cover the nose and mouth of a user. The oxygen and aerosol supply ports may be integrated or otherwise combined into a single port. Oxygen may be supplied from ambient air, from an oxygen tank, from an oxygen concentrator, or another medical device source. Aerosols are supplied from a nebulizer to the aerosol supply port, and respiratory products exhaled by the user are received from the exhaust port. A high efficiency filter (e.g., with a viral/bacterial filter alone, optionally combined with a hygroscopic condenser humidifier), and a fan are arranged downstream of the exhaust port. The fan creates a pressure differential that causes the respiratory products (e.g., aerosols) to flow through the filter, in which aerosols are captured, thereby reducing or eliminating the release of contaminated aerosols.

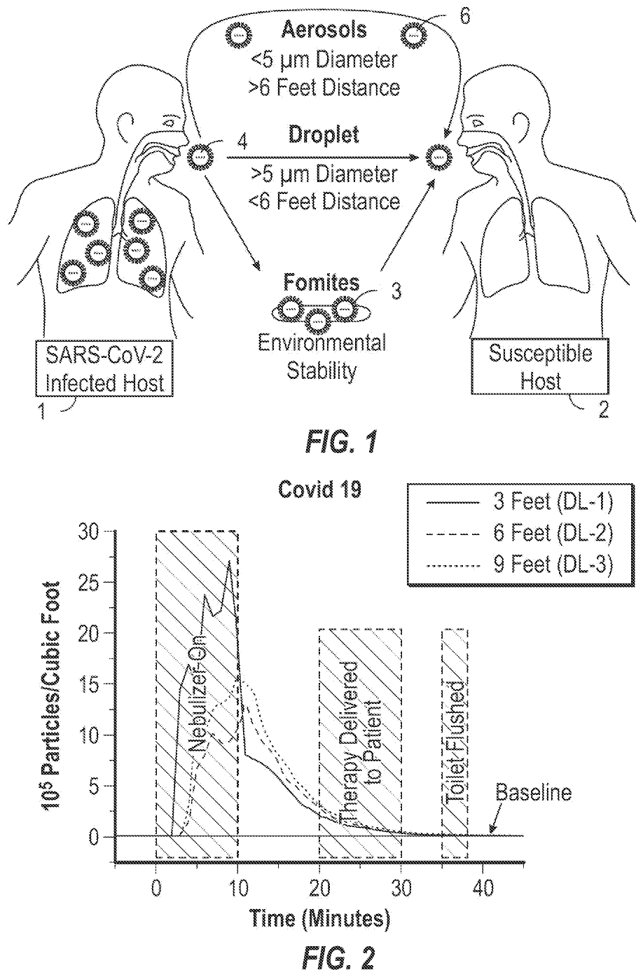

FIG. 1 is an illustrative diagram showing how droplets and aerosols may be propagated from an infected host 1 (e.g., a SARS-CoV-2 infected host) to a susceptible host 2 in an environment, and how environmentally stable fomites 3 derived from droplets 4 and/or aerosols 6 may accumulate within surfaces of the environment. FIG. 1 is reproduced from the following source: T. Galbadage, B. M Peterson, R. S. Gunasekera, Does COVID-19 spread thorugh droplets alone? Front. Public Health, 24 Apr. 2020, available online at URL:<https://doi.org/10.3389/fpubh.2020.00163≤.

FIG. 2 is a plot of aerosol particle concentration versus time showing aerosol particle counts per cubic foot transient over time, sensed at three different distances (3, 6, and 9 feet, respectively) from a patient undergoing nebulizer therapy. The measurement was carried out in a pre-operation room of 17 feet×13.6 feet×9 feet, equipped in the back with a small bathroom of 4 feet×6 feet 3 inches×9 feet 3 inches and an air ventilation rate of 20 changes per hour. The measurement was performed with a total of 3 sensors located at 3 feet (DL-1), 6 feet (DL-2) and 13 feet (DL-3) distance from the subject. The sensors were able to detect aerosol particles of 0.19 um or larger, and the sensor nominal setting for 0.5 um detection was used to account for the lower size aerosol particles. A subject was ventilated through high flow nasal cannula (HFNC) at 60 L/min. A nebulization therapy and an exercise therapy with respiratory therapy were performed during the measurement.

The “nebulizer on” portion of FIG. 2 shows particle count concentration during nebulization treatment of a patient under oxygen therapy using a high flow nasal cannula with 21% oxygen at 60 L/min and nebulization with 2.7 ml of albuterol and 0.3 mL of saline physiological solution delivered through a piezoelectric-based nebulizer. Comparative particle count profiles are illustrated for a COVID-19 infected patient at positions of 3 feet, 6 feet, and 9 feet from the subject, with the subject not wearing any mitigation mask.

Significant particle counts per cubic foot (feet³) were detected above the baseline level of the room, typically at about 7,400 counts/feet³ (for >0.2 um size count). During nebulization, the peak level was 365 times higher than the baseline level, and the particles were cleared out from the room after 20 min of completing the nebulization. In addition, it was observed that a proactive respiratory therapy executed by a therapist and including the subject performing breathing exercises at different conditions did not produce detectable aerosol-particle counts. Further, the action of flushing the bathroom toilet did not produce a particle count on the closest particle counter located at 6 feet away from the subject. This was possibly because the restroom even with a door open had its own ventilation system with air exchange rate of 20+ changes hour. Another notable feature was the spatial distribution of the aerosol on the room, although the closest location to the patient was the highest affected, the aerosol concentration peak at 9 feet location was one third larger than the corresponding peak at 6 feet location. The spread of potentially contaminated aerosol was therefore evident and its distribution was unpredictable.

In order to introduce solutions to the aerosol dispersion problem shown in FIG. 2, efficacy of different methods was explored, with particular emphasis on methods that may be deployed in a simple manner in various sites to avoid the spread of harmful microbes such as SARS-CoV-2.

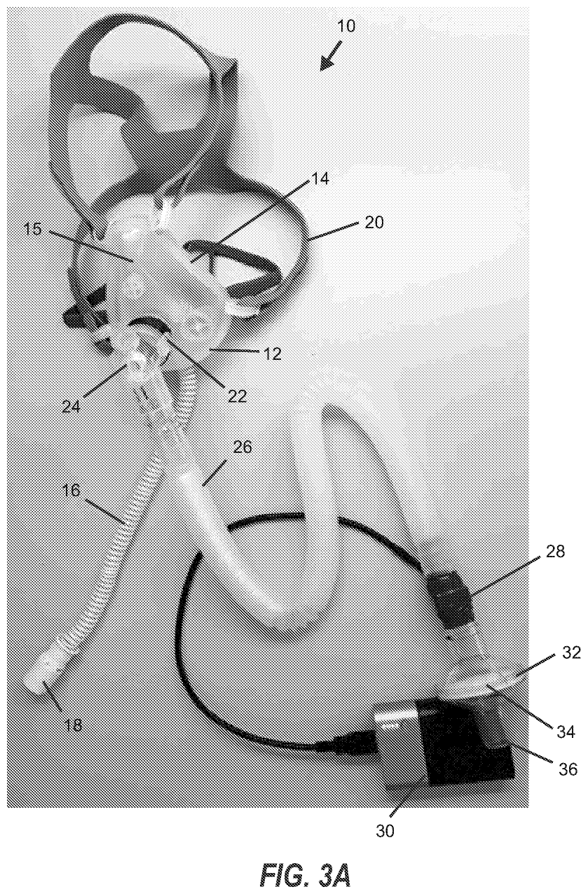

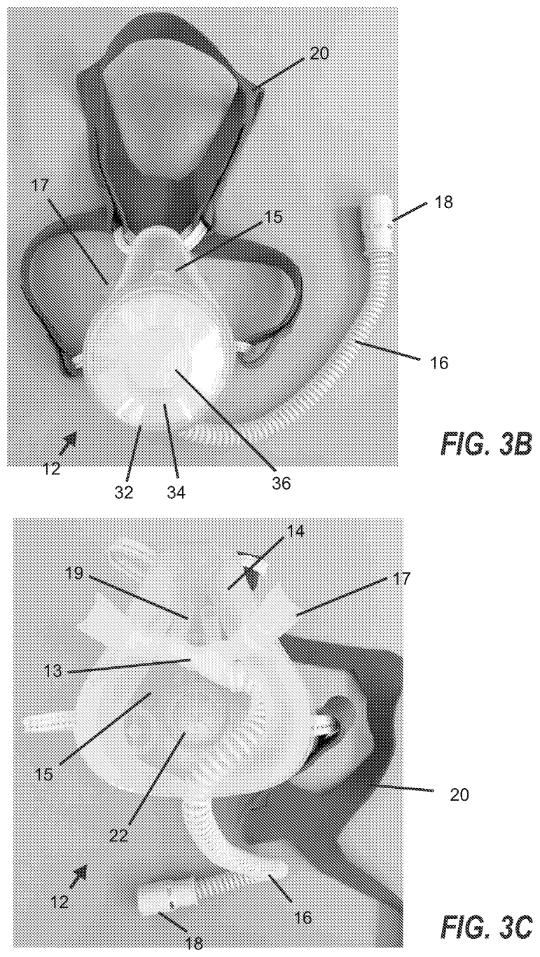

FIG. 3A is a front view of an aerosol mitigation apparatus 10 (e.g. useable for nebulization treatment) according to one embodiment of the present disclosure. The apparatus 10 includes a mask 12 having a compliant portion 14 (optionally formed of silicone) configured to conform to portions of the face of a user, and surrounding a body portion 15 (which may be substantially rigid, optionally formed of a light-transmissive polymeric material) that covers the nose and mouth of the user. The mask 12 may be low-cost and disposable. An aerosol supply hose 16 having a coupling 18 is arranged to be connected to an oxygen flow, and nebulizer (not shown) to supply oxygen and aerosols to the mask 12 through an oxygen and aerosol supply port (i.e., an opening connecting the mask 12 and the oxygen and aerosol supply hose 16). Head straps 20 coupled to the mask 12 are provided to maintain the mask 12 against the face of the user. An exhaust port 22 is located in the front portion of the mask 12 to permit the escape of respiratory products exhaled by a user wearing the mask 12. If needed, an optional check valve 24 may be provided and arranged proximate to the exhaust port 22. A flexible hose 26 may be arranged downstream of the exhaust port 22 to convey aerosols and respiratory products to an electrically operated fan 28, which may receive power from a portable battery pack 30. The fan 28 is configured to generate a pressure differential to create suction that draws respiratory products through the flexible hose 26 and forces the aerosols and respiratory products through a filter 34, which may have a hygroscopic condenser humidifier (HCH) 32 associated therewith. The filter 34 comprises a high efficiency filter (e.g., a HEPA filter or bacterial/viral filter) suitable for capturing aerosols with pathogens and preventing their release with gaseous respiratory products that exit the apparatus 10 through an outlet 36 coupled. Alternatively, the high efficiency filter 34 may be located before the fan 28 to prevent the fan from contamination and easier cleaning reprocessing.

FIGS. 3B and 3C provide front and rear portions of a nebulization treatment apparatus including a mask 12 according to the design shown in FIG. 3A, modified to provide a nasal cannula 17 (having nasal supply tubes 19) that is coupled with the aerosol supply hose 16 at a junction 13 that may serve as an aerosol supply port, and omitting the flexible hose 26 of FIG. 3A. The remaining elements of the mask 12 are generally in accordance with the mask 12 shown in FIG. 3A and are therefore not described again.

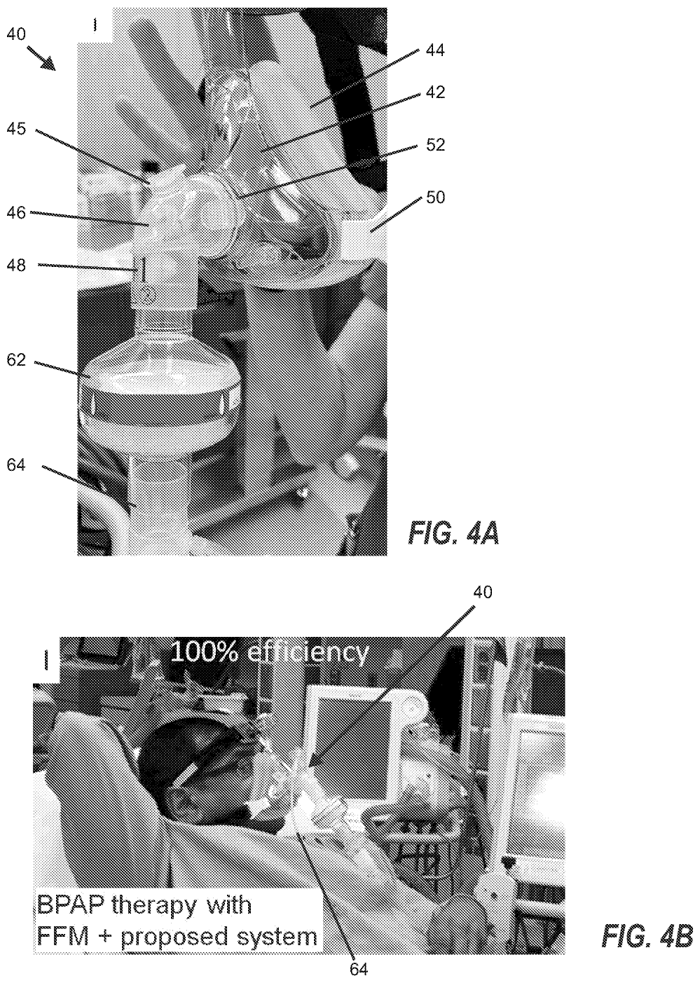

FIG. 4A depicts a nebulization treatment apparatus 40 including a full-face mask 42 with a HCH 62 having an integrated bacterial/viral filter as part of a bilevel positive airway pressure (BPAP) device according to one embodiment of the present disclosure. The face mask 42 includes a compliant portion 44 configured to conform to portions of a face of a user. Straps 50 are provided to fit the face mask 42 to a head of a user. The face mask 42 includes a frontward opening 52 to which a T-joint 46, with an aerosol supply port 45 (at top) and exhaust port 48 (bottom) arranged at two legs of the T-joint 46. (Although a T-joint may resemble the letter “T” in certain embodiments, the term as used herein is not so limited, and refers to any joint having one inlet and two outlets, or vice-versa, in any suitable geometric configuration). A check valve and/or internal partitions may be integrated into the T-joint 46 to prevent aerosols supplied to the aerosol supply port 45 from bypassing the face mask 42 and flowing directly to the outlet 48. Downstream of the T-joint 46, a HCH 62 with an integrated high efficiency filter is provided to receive respiratory products exhaled by the wearer of the face mask 42 and direct them to a HCH outlet 64. A fan (not shown) is arranged downstream (or upstream) of the HCH 62 and filter to cause aerosols and respiratory products to flow through the HCH 62, filter, and fan. FIG. 4B depicts a human test subject wearing the nebulization treatment apparatus 40 of FIG. 4A, with a supply hose 65 coupled thereto. Efficacy of the apparatus of FIGS. 4A-4B in mitigating aerosol release is evaluated as Case I representing a first one of five study cases described hereinafter.

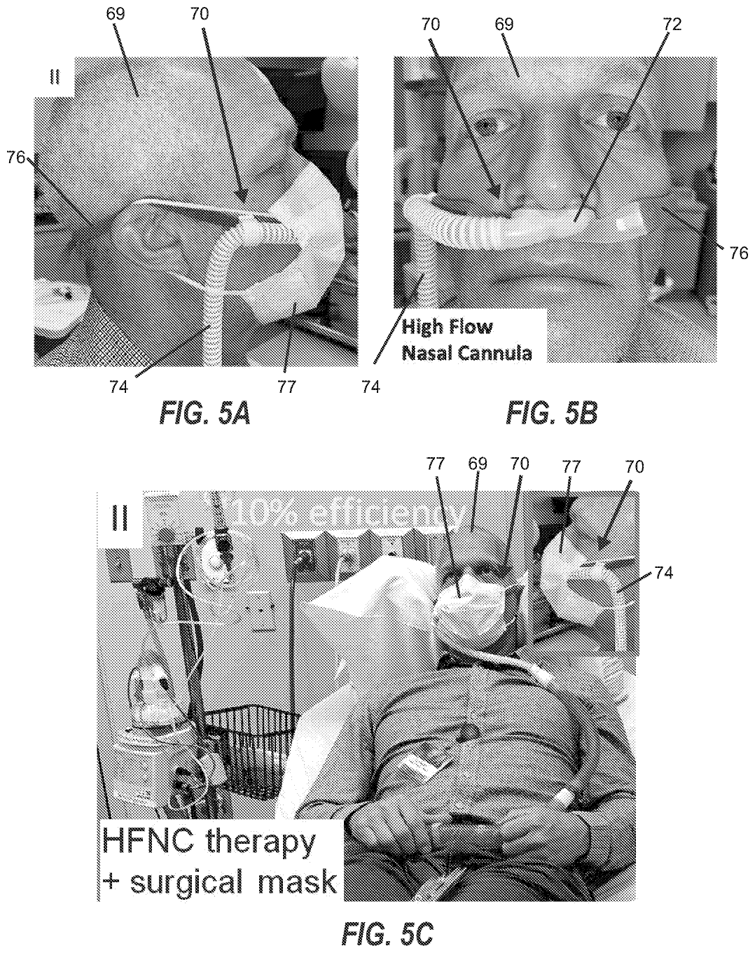

FIG. 5A is a side view of a human test subject 69 wearing a High Flow Nasal Cannula (HFNC) apparatus 70, including a gas supply hose 74 and a nasal supply fitting 72, useful as a nebulization treatment apparatus and covered by a surgical mask 77. FIG. 5B is a front view of the human test subject 69 and HFNC apparatus 70 of FIG. 5A, without presence of the surgical mask. FIG. 5C is a front view (and includes an inset left side view) of the human test 69 subject wearing the HFNC apparatus 70 and surgical mask 77 shown in FIG. 5A. Efficacy of the HFNC apparatus 70 and mask 77 of FIGS. 5A and 5C in mitigating aerosols is evaluated as Case II, representing a second one of five study cases described hereinafter.

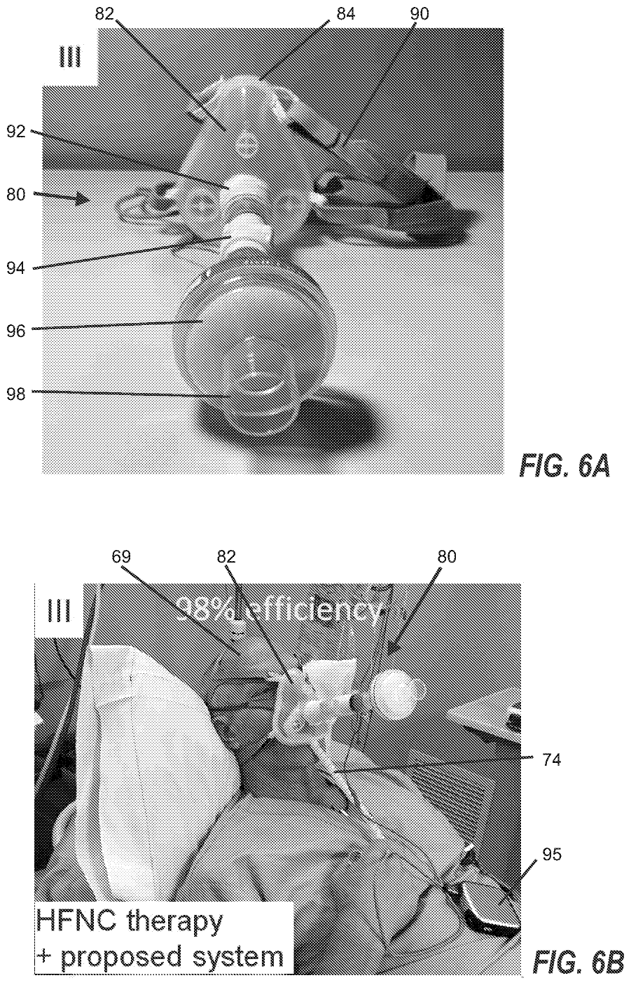

FIG. 6A is perspective view of at least a portion of a nebulization treatment apparatus 80 according to one embodiment including a silicone face mask 82 (having a compliant portion 84) modified to incorporate a nasal cannula according to the design shown in FIGS. 3B and 3C. Straps 90 are provided to affix the face mask to a head of a user. The face mask 82 has a single frontward opening 92 used as an outlet. An electrically operated fan 94 and a HCH 96 (having an integrated filter) are serially arranged downstream of the frontward opening 92, wherein the fan 94 serves to draw respiration products from the frontward opening 92 and eject them through the HCH and an HCH outlet 98. FIG. 6B is a perspective view of a human test subject 69 wearing the nebulization treatment apparatus 80 of FIG. 6A, showing a supply hose 74 for a cannula worn by the test subject under the mask 92, and showing a portable battery pack 95 for powering the fan of the apparatus 80. Efficacy of the apparatus 80 of FIGS. 6A-6B in mitigating aerosols is evaluated as Case III, representing a third one of five study cases described hereinafter.

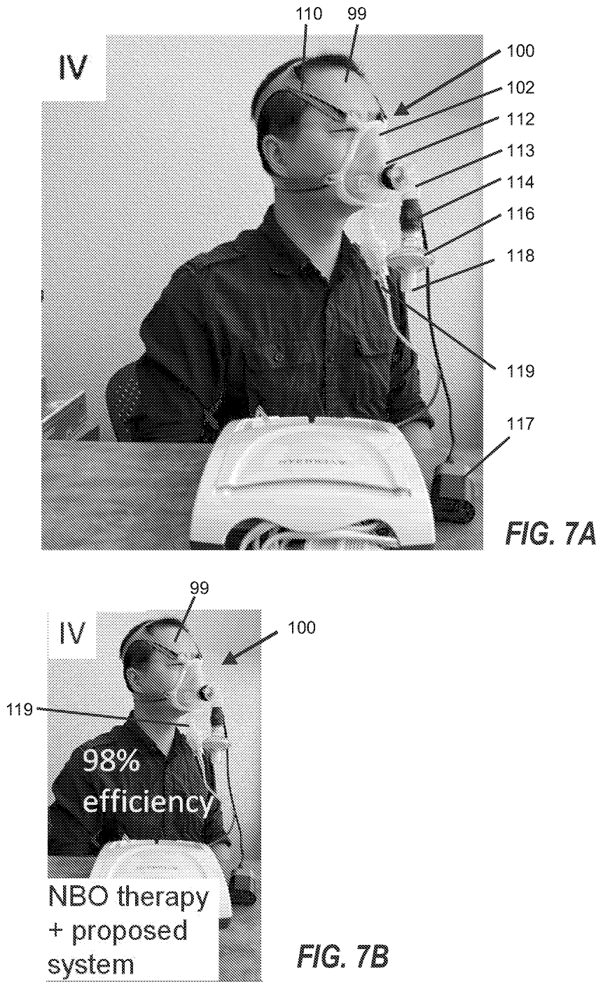

FIGS. 7A and 7B provide perspective views of a subject 99 connected to a nebulization treatment apparatus 100 according to one embodiment corresponding generally to the design shown in FIG. 3A, but without a flexible hose (26 in FIG. 3A) arranged between an electrically operated fan 114 and an exhaust port of the face mask 102. The subject 99 receives the oxygen supplied by a tube 119 through an oxygen inlet port provided by a one-way valve 113 on the face mask 102 (with the inlet port being next to a mask outlet port 112). The face mask 102 was devoid of a nasal cannula. The face mask 102 included a compliant portion 104 to confirm to a face of the user 99 and straps 110 to couple the face mask 102 to the user's head. Downstream of the one-way valve 113, an electrically operated fan 114 is arranged to draw respiration products from the face mask 102 and supply them to a downstream HCH 116 having an integrated high efficiency filter, and to eject filtered respiration products through a HCH outlet 118. A portable battery pack 117 is coupled with the electrically operated fan 114. Efficacy of the apparatus 100 of FIGS. 7A-7B in mitigating aerosols was evaluated as Case IV, representing a fourth one of five study cases described hereinafter.

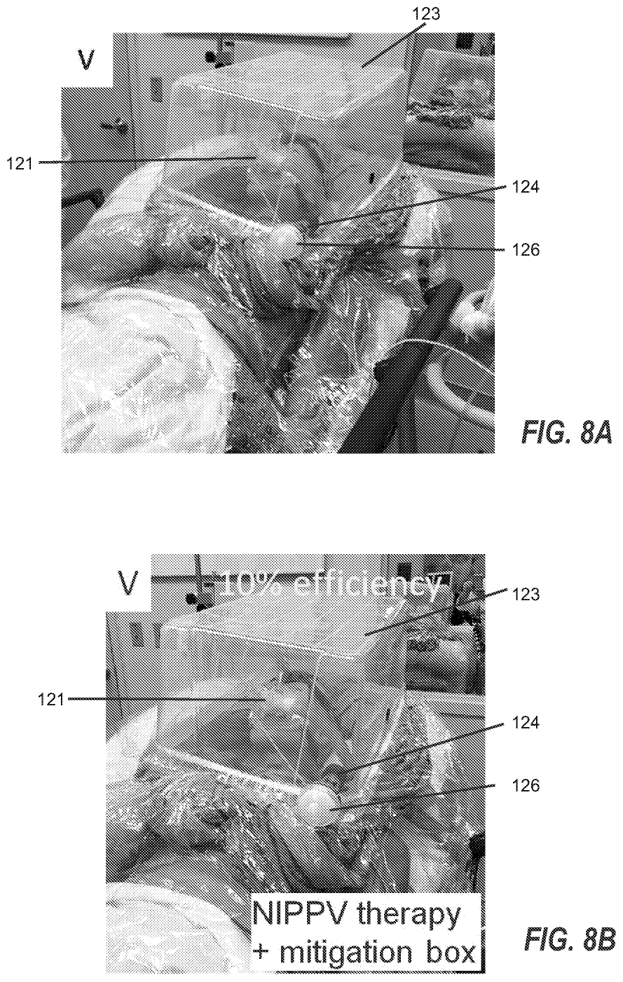

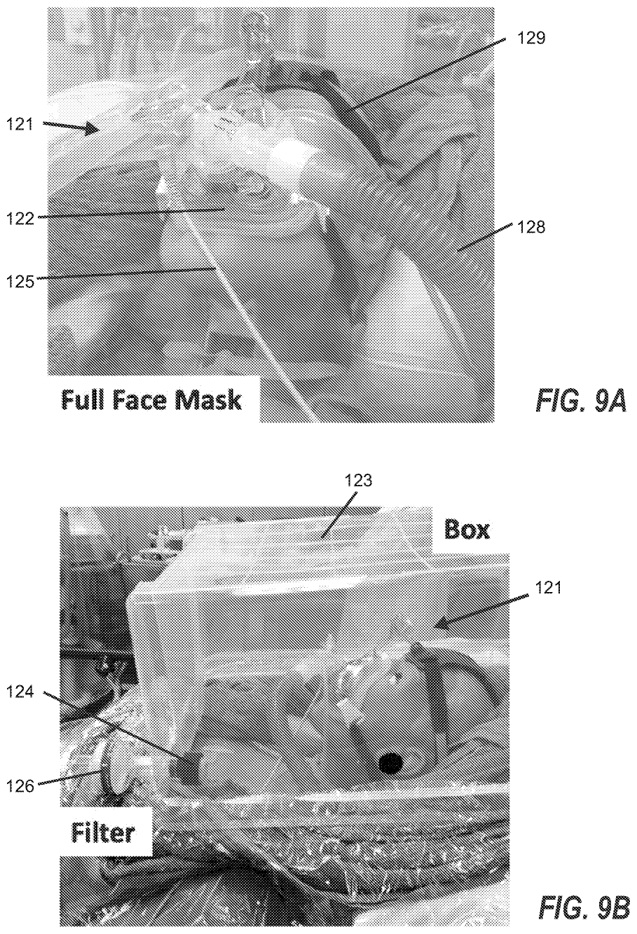

FIGS. 8A and 8B show a mannequin fitted with a Non-Invasive Positive Pressure Ventilator (NIPPV) 121, arranged in a mitigation box 123 having a bacterial/viral filter 126 arranged downstream of a fan 124 and coupled to a port defined in the mitigation box 123. FIG. 9A is a magnified front perspective view of a face mask portion 122 of the NIPPV 121, having an associated supply tube 125 and exhaust tube 128, affixed by straps 129 over a head of the mannequin as shown in FIGS. 8A-8B. FIG. 9B is a side view of the face mask 121, mitigation box 123, fan 124,and filter 126 of FIGS. 8A-8B. Efficacy of the NIPPV 121 and mitigation box 123 apparatus of FIGS. 8A-8B and FIGS. 9A-9B in mitigating aerosols is evaluated as Case V, representing a fifth one of five study cases described hereinafter.

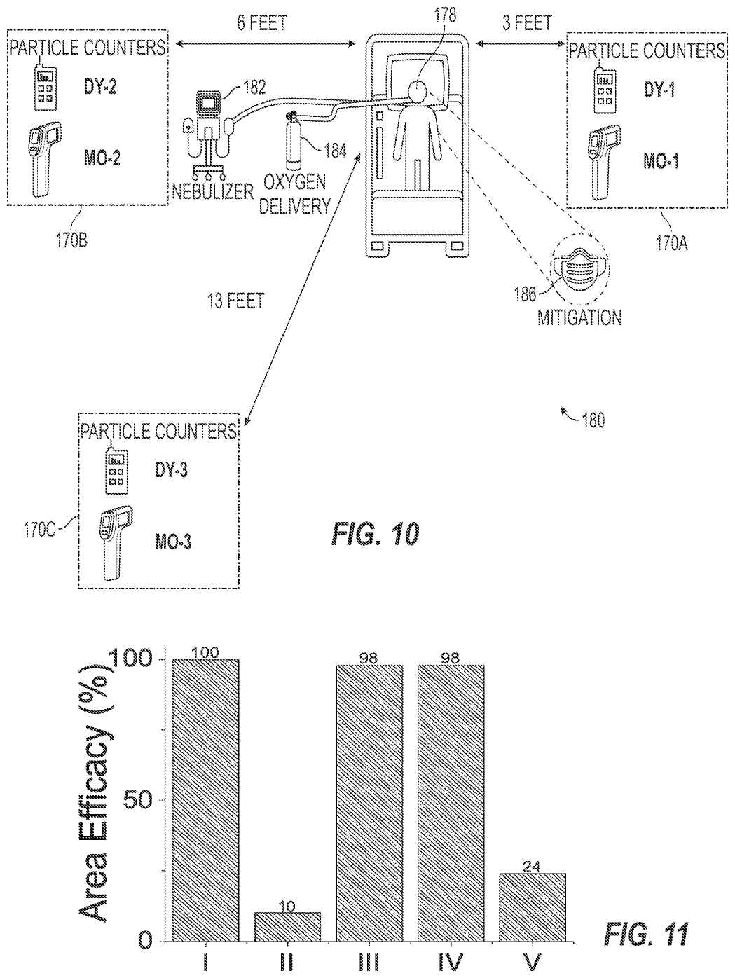

FIG. 10 is a diagram showing placement of sensing modules 170A (with components DY-1 and MO-1), 170B (with components DY-2 and MO-2), and 170C (with components DY3 & MO-3) having particle counters at different distances of 3, 6, and 13 feet, respectively from a patient or subject 178 undergoing nebulizer therapy via a nebulizer 182 incorporating oxygen delivery 184, wherein the patient 178 may have associated therewith exposure mitigation equipment 186 such as a mask, filter, and/or limited air exchange apparatus. Sensing modules 170A-170C are desirably placed at different locations in a conditioned environment 180, since contaminated aerosol concentration may vary considerably within the conditioned environment 180 due to flows of air generated by a HVAC or ventilation system.

FIG. 11 is a bar chart summarizing efficacy of different mitigation methods that were applied to the different oxygen therapies and the type of nebulizers according to Cases I to V as outlined hereinabove. Case I utilized the apparatus of FIGS. 4A-4B, embodying a BPAP device with a full face mask connected to a T-joint with a high efficiency filter and a fan. Case II utilized the apparatus of FIGS. 5A-5B, including a HFNC covered by a surgical mask. Case III utilized a silicone mask modified to incorporate a nasal cannula, plus an electrically operated fan arranged between (i) an exhaust port of the mask and (ii) and integrated high efficiency filter (yielding a “mitigation mask”), as illustrated in FIGS. 6A-6B. Case IV utilized a silicon mask with an electrically operated fan arranged between (i) an exhaust port of the mask and (ii) an integrated high efficiency filter (yielding another “mitigation mask”), as illustrated in FIGS. 7A-7B. Case V utilized the NIPPV and mitigation box with bacterial/viral filter as illustrated in FIGS. 8A-8B and FIGS. 9A-9B.

For Case I, an experiment was performed in the lab equipped with total four sensors—two sensors MO-1 and DY-1 at 3 feet distance, one sensor MO-2 placed at 6 feet and one sensor MO-3 at 13 feet from the test subject. Human-A was chosen as the test subject and was ventilated through a full face mask (FFM) with BPAP. The nebulization therapy of 3 ml saline physiological solution was delivered through a piezoelectric-based nebulizer. The subject wore the FFM and a filter and (downstream) fan to mitigate aerosol release.

For Case II, an experiment was carried out in the lab equipped with a total of four sensors—two sensors MO-1 and DY-1 at 3 feet distance, one sensor MO-2 placed at 6 feet and one sensor MO-3 at 13 feet from the test subject. Human-B was chosen as the test subject and was ventilated through HFNC. The nebulization therapy of 3 ml saline physiological solution was delivered through a piezoelectric based nebulizer. The subject wore a surgical mask to mitigate aerosol release.

For Case III, an experiment was carried out in the lab equipped with total six sensors—two sensors MO-1 and DY-1 at 3 feet distance, two sensors MO-2 and DY-2 at 6 feet and two sensors MO-3 and DY-3 at 13 feet from the test subject. Human-B was chosen as the test subject and was ventilated through HFNC. The nebulization therapy of 3 ml saline physiological solution was delivered through a piezoelectric based nebulizer. The subject wore a modified mitigation mask to mitigate aerosol release.

For Case IV, an experiment was carried out in a test lab equipped with total six sensors—two sensors MO-1 and DY-1 at 3 feet distance, two sensors MO-2 and DY-2 at 6 feet and two sensors MO-3 and DY-3 at 13 feet from the test subject. Human-C was chosen as the test subject and was breathing naturally without the help of any medical ventilators. The nebulization therapy of 3 ml saline physiological solution was delivered through a mechanical pump nebulizer. The subject wore a mitigation mask to mitigate aerosol release.

For Case V, an experiment was carried out in the simulation lab equipped with a total of six sensors—two sensors MO-1 and DY-1 at 3 feet distance, two sensors MO-2 and DY-2 at 6 feet and two sensors MO-3 and DY-3 at 13 feet from the test subject. A mannequin (medical model) was chosen as the test subject and was ventilated through a FFM with NIPPV. The nebulization therapy of 3 ml saline physiological solution was delivered through a piezoelectric based nebulizer. The model wore the mask and the box mitigation to mitigate aerosol release.

As it can be observed in FIG. 11, favorable mitigation solutions were found for Case I, Case III, and Case IV with a reduction of aerosol particles of 100%, 98% and 98% respectively—independently as to whether the efficacy was counted from the peak or the area under the curve of particle count concentration transients.

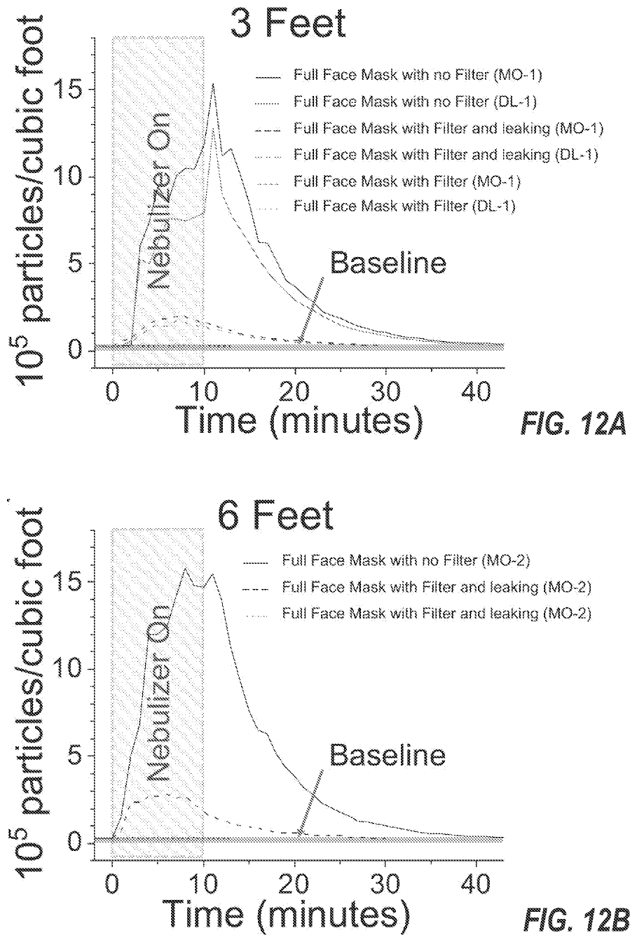

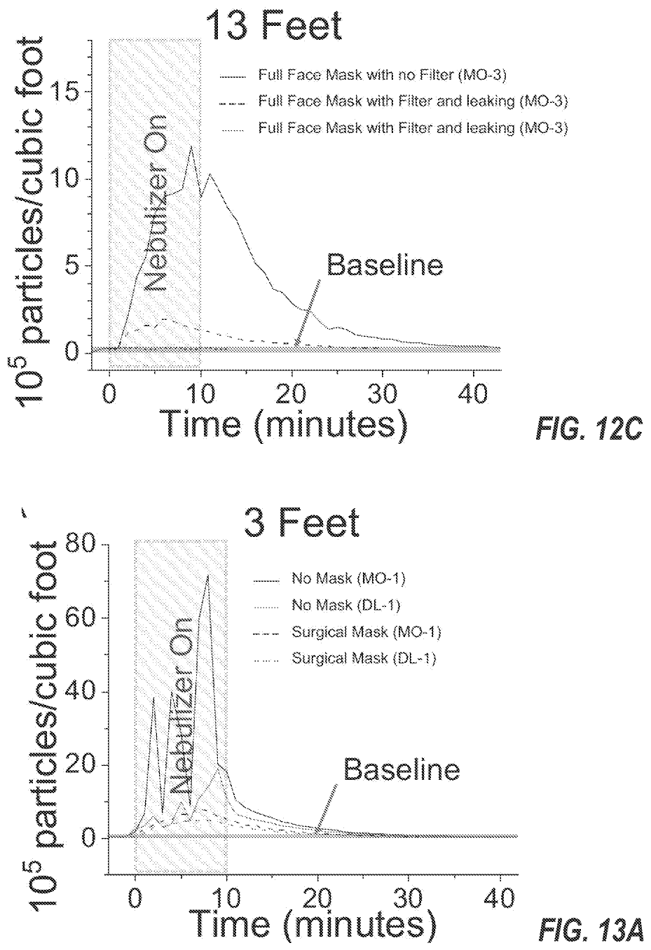

FIGS. 12A to 12C provide plots of aerosol particles per cubic foot versus time sensed at different distances from the subject for Case I. FIG. 12A provides plots from two sensors arranged three feet from the subject for three different experimental conditions, namely: use of a full face mask with no filter; use of a full face mask with a filter, wherein the mask is leaking; and use of a full face mask with filter (i.e., without mask leakage). FIGS. 12B and 12C provide plots from a single sensor arranged six feet and thirteen feet, respectively, from the subject for the same three experimental conditions described in connection with FIG. 12A.

In Case I, it is noteworthy that the use of a HEPA HME filter with 99.99% bacterial and viral filtration was 100% efficient, as long as the full-face mask was located on the subject's face with no leakage. However, any leak originating from incorrect use of the full-face mask was observed to cause significant release of particles to the room environment.

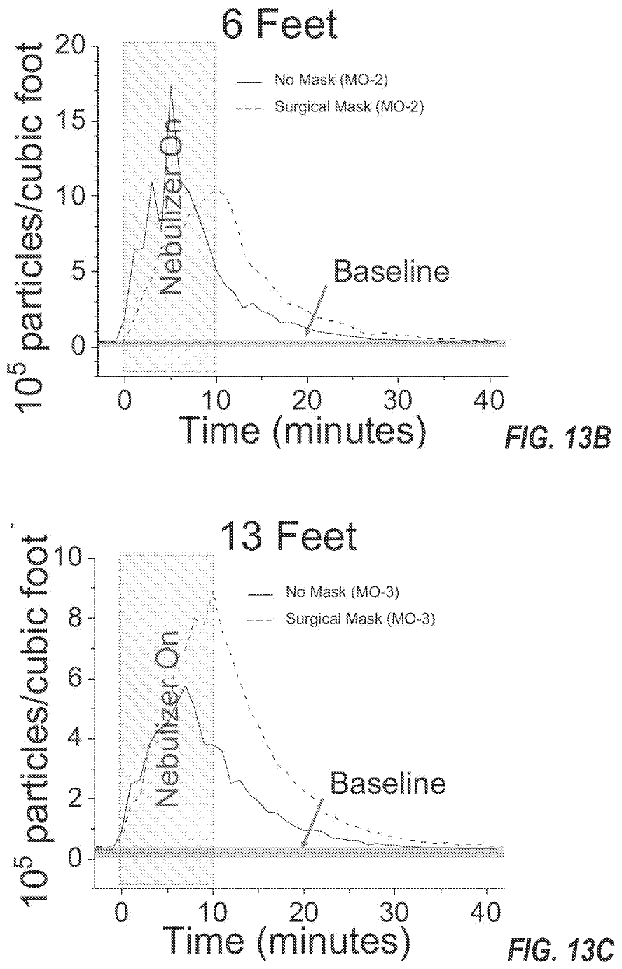

FIGS. 13A to 13C provide plots of aerosol particles per cubic foot versus time sensed at different distances from the subject for Case II. FIG. 13A provides plots from two sensors arranged three feet from the subject for two different experimental conditions, namely: use of a nasal cannula without mask and use of a nasal cannula covered with a surgical mask. FIGS. 13B and 13C provide plots from a single sensor arranged six feet and thirteen feet, respectively, from the subject for the same two experimental conditions described in connection with FIG. 13A.

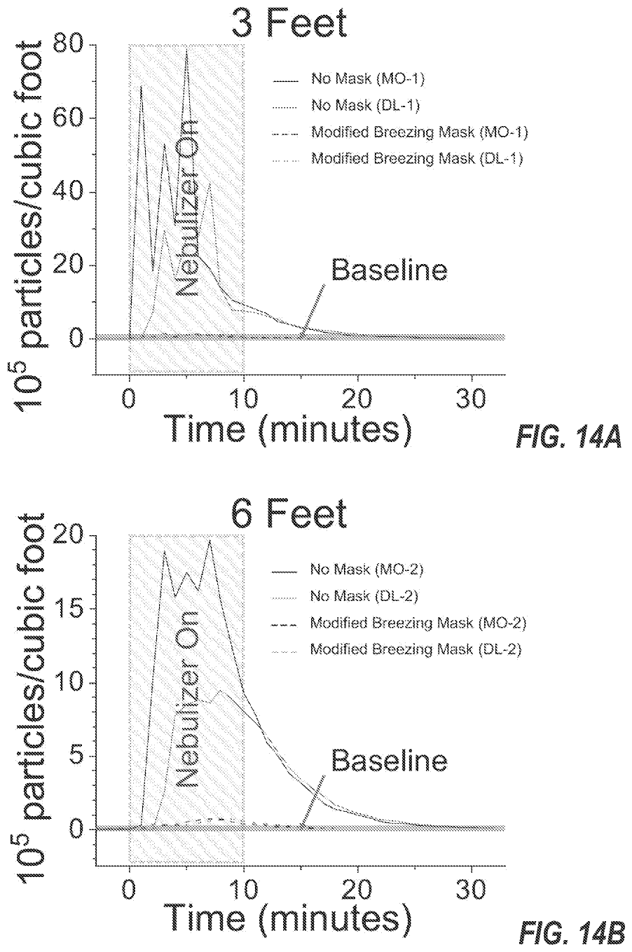

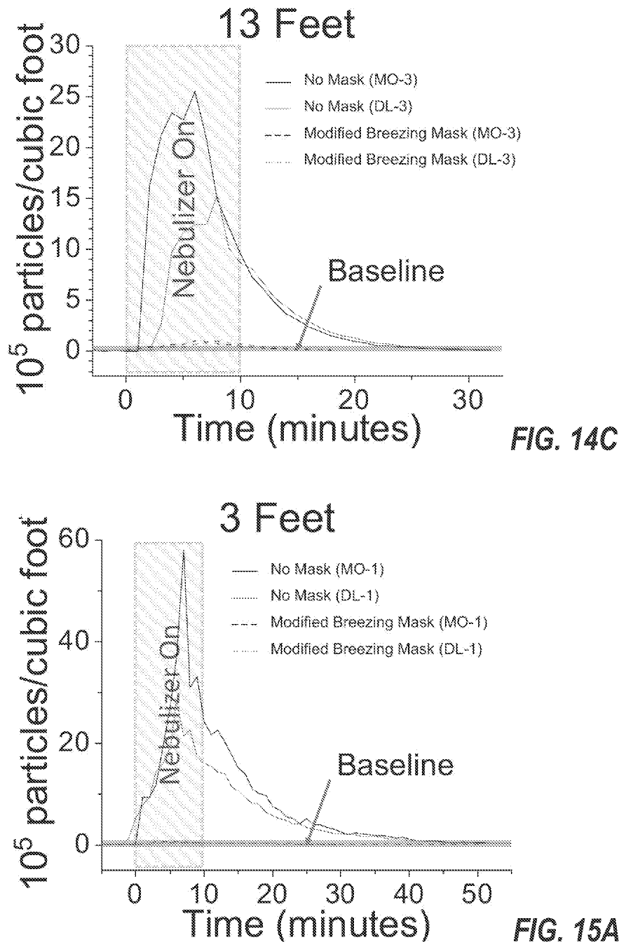

FIGS. 14A to 14C provide plots of aerosol particles per cubic foot versus time sensed at different distances from the subject for Case III. FIG. 14A provides plots from two sensors arranged three feet from the subject for two different experimental conditions, namely: use of a nebulizer with no mask, and use of a mitigation mask (named modified Breezing mask). FIGS. 14B and 14C provide plots from two sensors arranged six feet and thirteen feet, respectively, from the subject for same two experimental conditions described in connection with FIG. 14A.

In Case II and Case III, it noteworthy that several baseline studies were conducted including in absence of a nebulizer. It was shown that while a subject is using the nasal cannula and no coughing, talking or sneezing, regardless the magnitude of the flow 30 or 60 L/min, the fact of wearing or not wearing a surgical mask did not affect the particle counting, which remained within typical baseline levels for the room. In this case, the room was a simulation operating room which consistently had <50,000 particles per cubic foot. This is an interesting finding since it shows the nebulization therapy seemed to be an important factor of aerosol generation. In addition, this therapy has been the option of choice at Mayo Clinic since the use of NIPPV has shown detrimental outcomes in fatality rates. However, the high counts generated by the nebulizer could not be mitigated efficiently by the use of a surgical mask according to Case II. On the contrary, efficient mitigation for this could be achieved with almost 100% efficacy by using the mitigation mask solution presented in this disclosure, which is so-called modified “Breezing” mask. An original “Breezing” mask would usually be used for assessing resting metabolic rate (i.e., Breezing Pro). The modified mask has a silicone soft edge that allowed the introduction of a nasal cannula and nebulizer hose, and enabled tight sealing onto the face, using a customized mask strap. In addition, the mask was modified with a high efficiency filter, and a fan that promoted withdrawal of aerosols, respiration products, including the exhaled and unused therapeutic air from the high flow therapy.

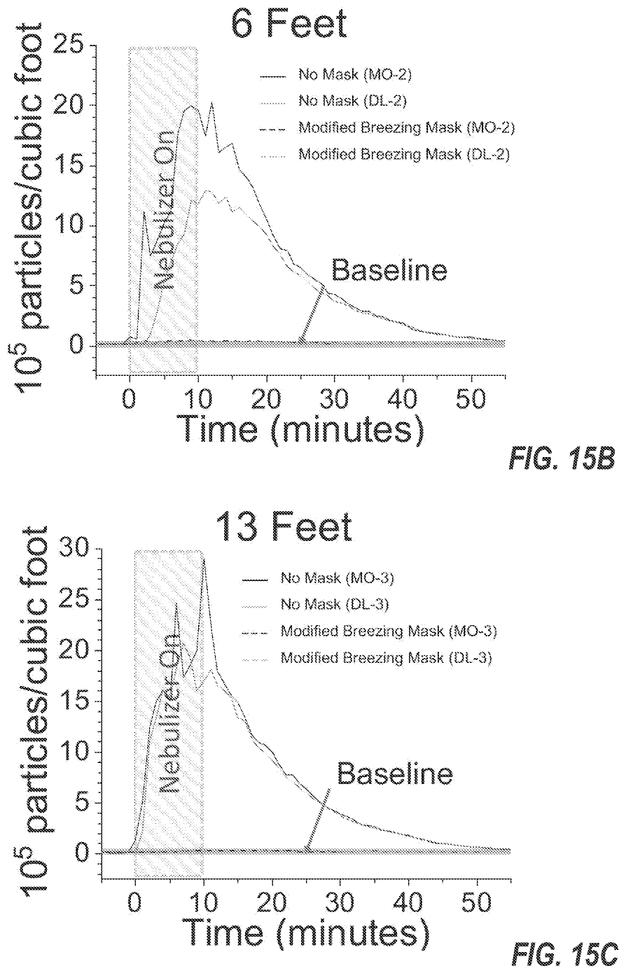

FIGS. 15A to 15C provide plots of aerosol particles per cubic foot versus time sensed at a different distances from the subject for Case IV. FIG. 15A provides plots from two sensors arranged three feet from the subject for two different experimental conditions, namely: use of a nebulizer with no mask, and use of a mitigation mask solution (modified Breezing mask). FIGS. 15B and 15C provide plots from two sensors arranged six feet and thirteen feet, respectively, from the subject for same two experimental conditions described in connection with FIG. 15A.

Case IV involved the use of the mitigation mask solution (modified “Breezing” mask) to mitigate aerosol released from a regular nebulizer therapy using ambient air from the room. The oxygen was inhaled via an oxygen port (one-way valve), which enabled ambient air inhalation under normal breathing conditions. The nebulizer delivered a 4 L/min stream of air with the aerosol through an aerosol supply port. The modified mask used in Case IV mitigated the nebulizer's aerosol dispersion with 100% efficiency.

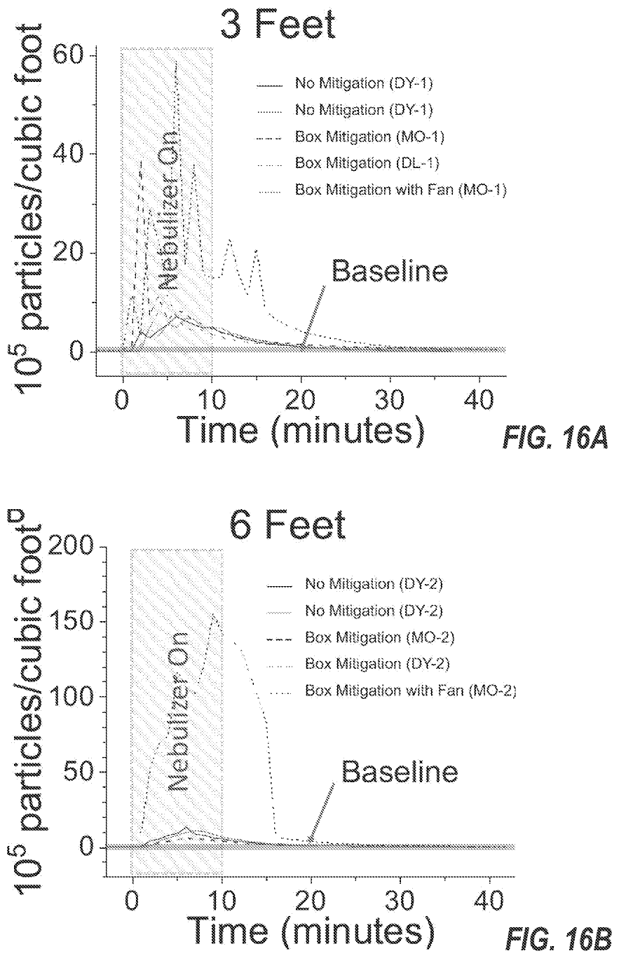

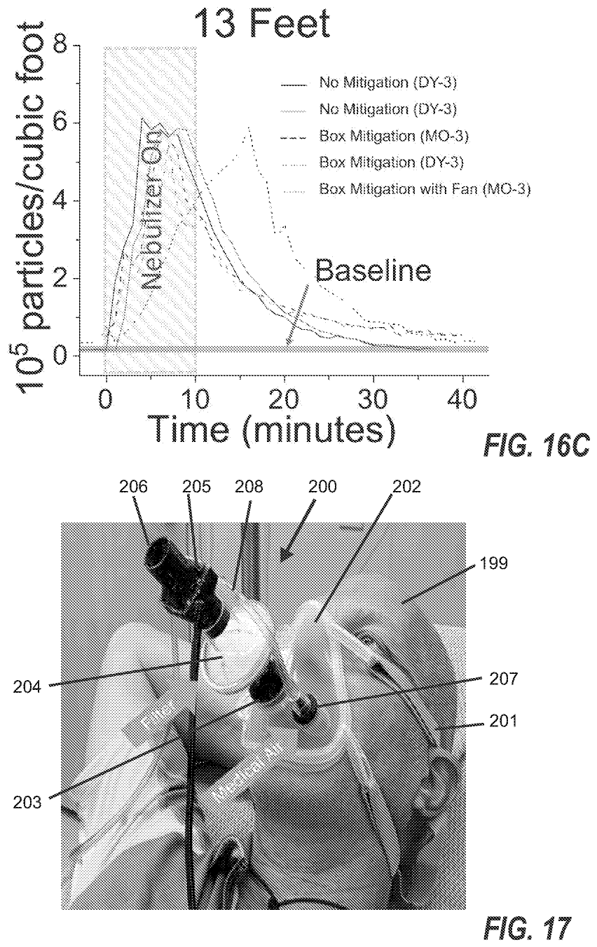

FIGS. 16A to 16C provide plots of aerosol particles per cubic foot versus time sensed at different distances from the subject for Case V. FIG. 16A provides plots from two sensors arranged three feet from the subject (model) for three different experimental conditions, namely: use of a nebulizer with no mitigation, and use of a nebulizer with box mitigation with a filter. FIGS. 16B and 16C provide plots from two sensors arranged six feet and thirteen feet, respectively, from the subject (model) for same experimental conditions described in connection with FIG. 16A.

Case V, which was conducted with a mannequin model, entailed a leak on the face mask of about 35 L/min. This type of leak was typical for most patients. In this case, the mitigation box was ineffective at preventing aerosol release—despite the box being tightly attached to the model using wet towels surrounding the edges of the box, which were further covered with 2 mil thickness plastic covers sealed to the box. The box was provided with a HEPA filter. The inefficacy of this method was significant, particularly given that mitigation boxes have been previously recommended for reducing aerosol release.

From the above-described series of experiments, it is evident that use of surgical masks (Case II) and box mitigation with a stand-alone filter only (Case V) are not recommended for mitigating release of aerosols to address an airborne disease like SARS-Cov-2. The best solution for such a scenario would be to employ a mask with a high efficiency filter and an appropriately designed fan; however, such masks and/or filters may be in high demand and not readily accessible during a pandemic. A mask as described and claimed herein therefore represents an attractive alternative to mitigate release of aerosols.

FIG. 17 shows an aerosol mitigation apparatus 200 including a mask 202, designed for transportation of a patient 199, who can be a potential source of contaminated aerosols via coughing, sneezing, or using the vocal cords (e.g., talking, singing, etc.). The aerosol mitigation apparatus 200 includes a face mask 202 with associated straps 201, the mask 200 having an inlet port 207 (for receiving a gas or aerosol supply tube 208) and an outlet port 203. A high efficiency filter 204 (which may be integrated in, or associated with, a hygroscopic condenser humidifier (HCH)) is arranged between the outlet port 203 and an electrically operated fan 205. The fan 205 is arranged to draw respiration products from the outlet port 203 and through the filter 204, so that filtered respiration products (e.g., devoid of aerosols generated by the patient 199) exit through a fan outlet 206. The supply tube 108 may be used to supply medical oxygen to the patient 199. The fan 205 may be powered by a portable battery pack (not shown). The aerosol mitigation apparatus 200 can be used while the patient 199 is transported or introduced inside an imaging system such as a CT scanner.

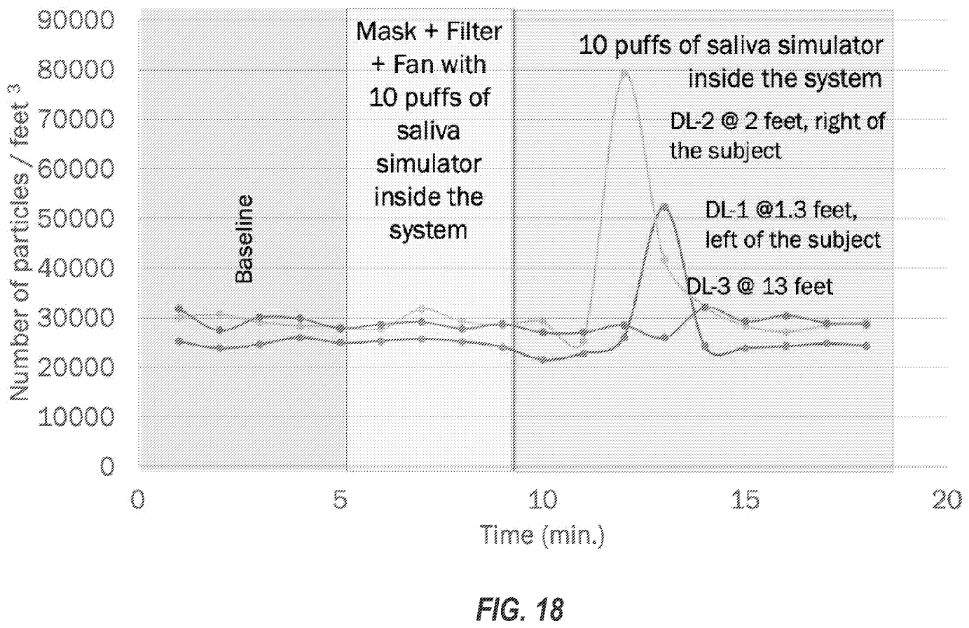

FIG. 18 provides plots of aerosol particles per cubic feet versus time sensed at different distances from a subject wearing a system like the one shown in FIG. 17. The sensors (DL) were installed at 1.5, 2, and 13 feet from the subject respectively. The sensors have a sensitivity to 0.2 um size particles. Simulation of coughing and sneezing was performed with 10 puffs of a sprayer inside and outside the system comprised of the mitigation mask with a filter and fan. The assembly of mask, filter, and fan enabled to mitigate 100% the dispersion of the aerosol particles.

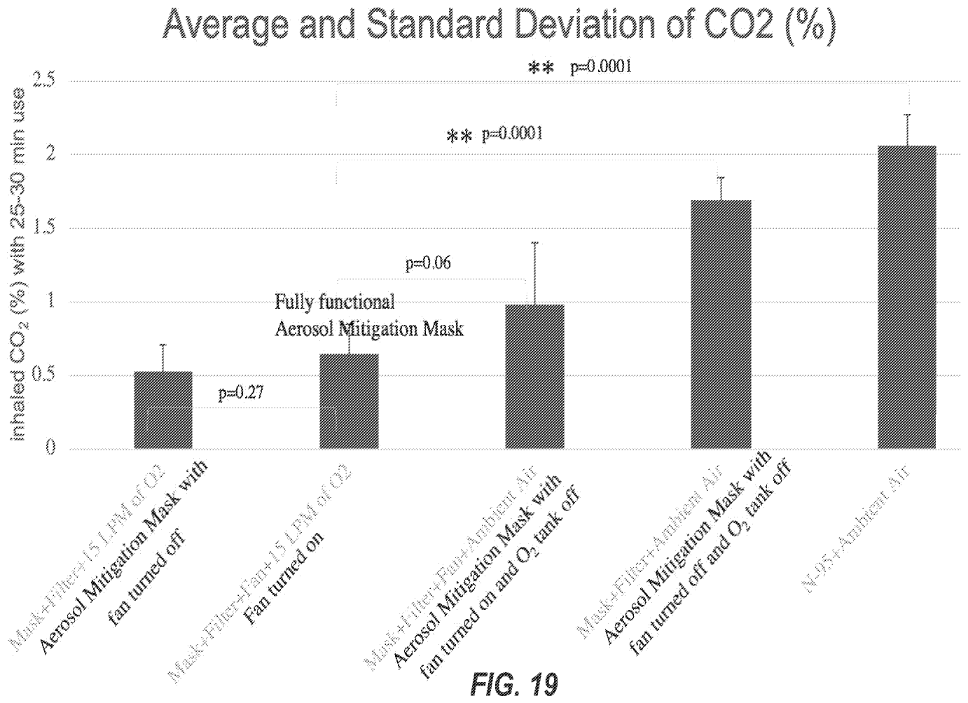

FIG. 19 shows the inhaled carbon dioxide levels of the aerosol mitigation mask system in FIG. 17 for different conditions, including fan on, fan off, and oxygen delivery off supplemented with ambient air via an emergency inhalation valve in comparison with the carbon dioxide levels of the N95 mask. It is evident that the fully functional aerosol mitigation mask brings better breathing conditions that a N-95 mask supplied with ambient air. Other conditions where one of the components of the aerosol mitigation mask system fails (fan or oxygen) also assured relatively safer conditions for the wearer.

It is important to mention that the characteristics of the fan are relevant and must sustain a flow compatible with the subject's breathing rate (e.g. breathing peak flow, frequency, end tidal volume and average exhalation rate) as well as the oxygen therapy's flow which is dependent on the type of oxygen delivery.

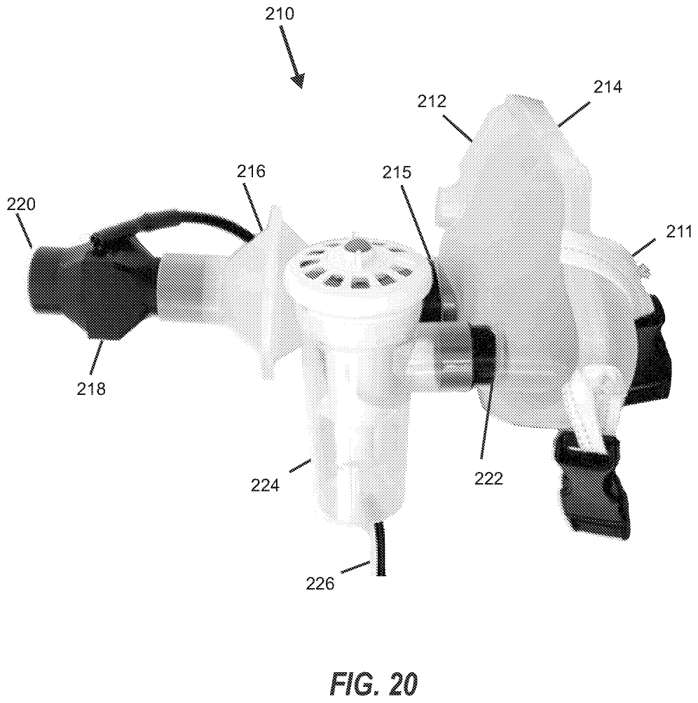

FIG. 20 shows a nebulization treatment apparatus 210 providing aerosol barrier utility, with the apparatus 210 including a face mask 212 having a compliant portion 214 and straps 211, as well as an outlet port 215 and an inlet port 222. The apparatus 210 includes a fan 218 (having a fan outlet 220) and a high efficiency filter 216 coupled to the outlet port 215, and includes a breath-actuated nebulizer 224 (with associated electrical wires 226) coupled with the inlet port 222 of the face mask 212. The breath-actuated nebulizer 224 is used to provide a nebulized drug in aerosol form, while the fan 218 is used to draw exhaled respiration products including aerosols through the filter 216 to capture any exhaled aerosols and prevent their release to an environment around the mask 212.

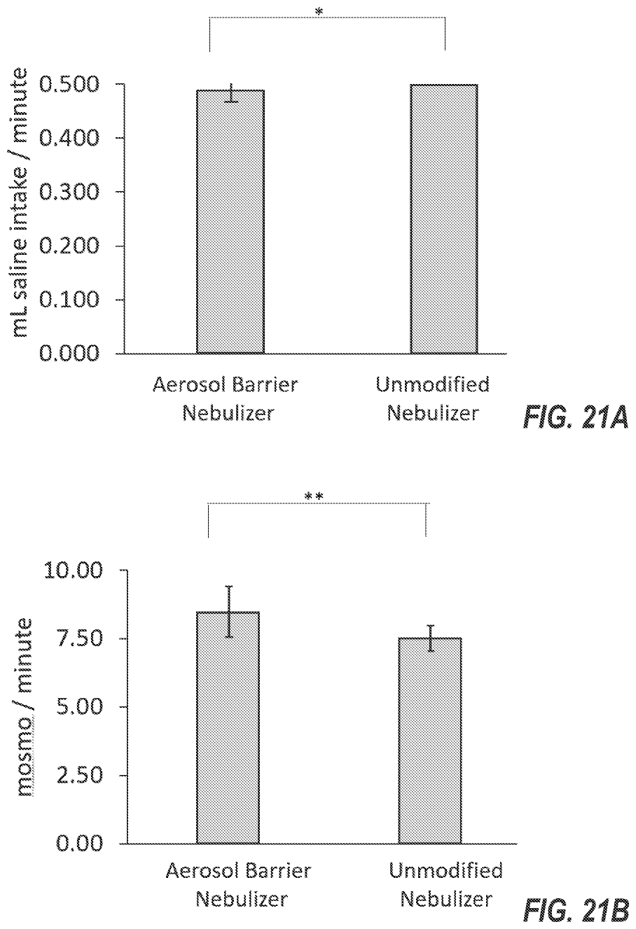

FIG. 21A is a bar chart comparing amounts of saline physiological solution intake (mL intake per minute) between an aerosol barrier nebulizer (N=7) and an unmodified nebulizer (N=9). The saline physiological solution was used as a drug simulant. Solution intake volume per minute was assessed in a nebulizer supply cup. FIG. 21B is a bar chart comparing absolute solution intake amount (in osmolarity units) per minute assessed in an inhalation filter Experiments were set with 3 mL of saline solution, 6 liters per minute oxygen flow, end tidal volume of 500 mL, and 15 breaths per minute. Referring to FIG. 21A, no statistically significant difference was found in the volume intake per minute between the aerosol barrier nebulizer and a conventional nebulizer. However, referring to FIG. 21B, a statistically significant difference was observed between the two nebulizers, in that the aerosol barrier nebulizer was slightly more efficient to deliver saline solution in the inhalation process. That is, the aerosol barrier nebulizer according to FIG. 20 can deliver the same or slightly greater amount of drug per minute than a conventional nebulizer. This demonstrates that the function of drug delivery by an aerosol barrier nebulizer is either unaltered or slightly improved compared to a conventional non-aerosol-barrier nebulizer, such that the aerosol barrier nebulizer offers efficacy of therapeutic treatment while adding safety in the reduction or elimination of aerosol spread.

It will be apparent to those skilled in the art that various modifications and variations can be made without departing from the spirit or scope of the invention.

Many modifications and other embodiments of the embodiments set forth herein will come to mind to one skilled in the art to which the embodiments pertain having the benefit of the teachings presented in the foregoing descriptions and the associated drawings. Therefore, it is to be understood that the description and claims are not to be limited to the specific embodiments disclosed and that modifications and other embodiments are intended to be included within the scope of the appended claims. It is intended that the embodiments cover the modifications and variations of the embodiments provided they come within the scope of the appended claims and their equivalents. Although specific terms are employed herein, they are used in a generic and descriptive sense only and not for purposes of limitation.

### REFERENCES

1. Maclean, S. Causes and spread of infection. in Level 2 Diploma In Health & Social Care 424-448 (2014).
2. Stadnytskyi, V., Bax, C. E., Bax, A. & Anfinrud, P. The airborne lifetime of small speech droplets and their potential importance in SARS-CoV-2 transmission. Proc. Natl. Acad. Sci. 117, 11875-11877 (2020).
3. Tran, K., Cimon, K., Severn, M., Pessoa-Silva, C. L. & Conly, J. Aerosol generating procedures and risk of transmission of acute respiratory infections to healthcare workers: a systematic review. PLoS One 7, (2012).
4. Liu, Y. et al. Aerodynamic analysis of SARS-CoV-2 in two Wuhan hospitals. Nature 1-4 (2020).
5. Baron, P. Generation and behavior of airborne particles (aerosols). (2010).
6. Howard, J. et al. Face masks against COVID-19: an evidence review. (2020).
7. Kaufman, A. Portable life support system. (1982).
8. Turak, N. Wearing a mask can significantly reduce coronavirus transmission, study on hamsters claims. (2020). Available at: https://www.cnbc.com/amp/2020/05/19/coronavirus-wearing-a-mask-can-reduce-transmission-by-75percent-new-study-claims.html?fbclid=IwAR1sSICZFohFW_bMW8_EFxqdvjY3GSGN9OrU3deMN2cyvYr EZbtHPPX0WEs. (Accessed: 10 Jun. 2020)
9. Santarpia, J. L. et al. Transmission potential of SARS-CoV-2 in viral shedding observed at the University of Nebraska Medical Center. MedRxIV(2020).
10. Zayas, G. et al. Cough aerosol in healthy participants: fundamental knowledge to optimize droplet-spread infectious respiratory disease management. BMC Pulm. Med. 12, 11 (2012).
11. Shaw, K. M. et al. Intensive care unit isolation hood decreases risk of aerosolization during noninvasive ventilation with COVID-19. Can. J. Anaesth. 1 (2020).
12. Simones, M. P. et al. Measurement of the size and charge distribution of sodium chloride particles generated by an Aeroneb Pro®pharmaceutical nebulizer. Eur. J. Nanomedicine 6, 29-36 (2014).
13. Procedural Safety Hood. (2020). Available at: https://bellmedical.com/stat-enclosure, 10 Jun. 2020

1. An aerosol mitigation apparatus comprising:
a mask comprising a compliant portion configured to conform to portions of a face of a user and cover a nose and mouth of the user;
at least one oxygen supply port associated with the mask and configured to provide oxygen to the mask for inhalation by the user;
an exhaust port associated with the mask and configured to receive respiratory aerosols emitted by the user;
a high efficiency filter arranged downstream of the exhaust port to receive and filter respiratory aerosols exhaled by the user; and
a fan configured to promote flow of respiratory aerosols from the exhaust port to the high efficiency filter.

2. The aerosol mitigation apparatus of claim 1, wherein the compliant portion comprises silicone.

3. The aerosol mitigation apparatus of claim 1, wherein the fan is configured to sustain a flow rate in a range of from 10 L/min to 800 L/min.

4. The aerosol mitigation apparatus of claim 1, wherein the at least one oxygen port includes an oxygen emergency port configured to admit ambient air for inhalation by the user.

5. The aerosol mitigation apparatus of claim 1, wherein the fan is arranged either (A) downstream of the high efficiency filter, or (B) between (i) the exhaust port and (ii) the high efficiency filter

6. The aerosol mitigation apparatus of claim 1, wherein the filter comprises a hygroscopic condenser humidifier as part of a hygroscopic condenser humidifier filter.

7. The aerosol mitigation apparatus of claim 1, further comprising a flexible hose extending between the exhaust port and the high efficiency filter.

8. The aerosol mitigation apparatus of claim 1, further comprising a portable battery pack or power supply coupled with the fan.

9. The aerosol mitigation apparatus of claim 1, further comprising an aerosol supply port associated with the mask and configured to supply aerosols from a nebulizer to the mask to be inhaled by the user.

10. The aerosol mitigation apparatus of claim 1, further comprising a breath actuated nebulizer associated with the mask and configured to supply aerosols to the mask to be inhaled by the user.

11. The aerosol mitigation apparatus of claim 1, further comprising a nasal cannula integrated with the mask and configured to receive oxygen and/or aerosols.

12. The aerosol mitigation apparatus of claim 1, further comprising a head strap coupled with the mask and configured to secure the mask to the face of the user without air leakage.

13. The aerosol mitigation apparatus of claim 1, further comprising a T-joint coupled with the mask, wherein the aerosol supply port and the exhaust port are arranged at two legs of the T-joint.

14. The aerosol mitigation apparatus of claim 1, wherein the high efficiency filter comprises a HEPA filter.

15. The aerosol mitigation apparatus of claim 1, wherein the high efficiency filter comprises a bacterial/viral filter.

16. A method for reducing aerosol release from a respiring user, the method comprising:
supplying oxygen from at least one supply port associated with a mask, wherein the mask comprises a compliant portion configured to conform to portions of a face of the user and cover a nose and mouth of the user, and the oxygen is to be inhaled by the user through the mask; and
receiving aerosol products exhaled by the user from an exhaust port associated with the mask, and creating a pressure differential using a fan to cause the aerosol products to flow to a high efficiency filter arranged downstream of the exhaust port.

17. A method for reducing aerosol release from a nebulizer treatment device, the method comprising:
supplying oxygen from at least one supply port associated with a mask, wherein the mask comprises a compliant portion configured to conform to portions of a face of a user and cover a nose and mouth of the user, and the oxygen is to be inhaled by the user through the mask;
supplying aerosols from a nebulizer to an aerosol supply port associated with a mask, wherein the mask comprises a compliant portion configured to conform to portions of a face of a user and cover a nose and mouth of the user, and the aerosols to be inhaled by the user through the mask; and
receiving aerosol products exhaled by the user from an exhaust port associated with the mask, and creating a pressure differential using a fan to cause the aerosol products to flow to a high efficiency filter arranged downstream of the exhaust port.

18. The method of claim 17, wherein the nebulizer comprises a breath actuated nebulizer associated with the mask.

19. The method of claim 16, wherein the high efficiency filter and the fan are coupled with the mask.

20. The method of claim 16, wherein the fan comprises a battery powered fan.
